(19) United States
(12) Patent Application Publication
(10) Pub. No.: US 2007/0173829 A1
(43) Pub. Date: Jul. 26, 2007

(54) Devices and methods for connecting vertebral rods

(75) Inventors: Troy D. Drewry, Germantown, TN (US); William Barry Null, Olive Branch, MS (US); Marc Paul, Memphis, TN (US)

Correspondence Address: COATS & BENNETT, PLLC, 1400 Crescent Green, Suite 300, Cary, NC, 27518, US

(73) Assignee: SDGI Holdings, Inc.

(21) Appl. No.: 11/337,947

(22) Filed: Jan. 23, 2006

(51) Int. Cl. A61F 2/30

(52) U.S. Cl. 606/61

(57) ABSTRACT

Connection devices and methods to connect together vertebral rods. Embodiments of the device include a first member and a second member. Each member may include a grip for connecting with the vertebral rods. A connector may connect together the first and second members. The connector provides for various manners of adjusting the position of the extension members. Connector may provide for relative rotation of the members about one or more axes. Connector and the construction of the device may also provide for adjustment of a length of the device.

### BACKGROUND

The present application is directed to devices to connect rods used in spinal fixation, and more specifically, to an adjustable connector having multiple degrees of freedom for connecting vertebral rods.

The spine is divided into four regions comprising the cervical, thoracic, lumbar, and sacrococcygeal regions. The cervical region includes the top seven vertebrae identified as C1-C7. The thoracic region includes the next twelve vertebrae identified as T1-T12. The lumbar region includes five vertebrae L1-L5. The sacrococcygeal region includes nine fused vertebrae that form the sacrum and the coccyx. The vertebrae of the spine are aligned in a curved configuration that includes a cervical curve, thoracic curve, and lumbosacral curve.

Vertebral rods may be implanted to support and position vertebrae in one or more of these regions. The rods extend along a section of the spine and are connected to the vertebrae with one or more fasteners. The rods may have a curved configuration to conform to the curvature of the spine. Often times two or more rods are connected together and work in combination to support and position the vertebrae. The rods may have the same or different shapes and sizes depending upon their position along the spine.

One or more connectors may attach the rods together for further stabilization and positioning. The connectors have a first connection to the first rod, and a second connection to the second rod. The contour of the spine often makes it difficult to position a connector to attach the rods together.

### SUMMARY

The application is directed to connection devices and methods to connect together vertebral rods. The device may include a first member and a second member. A grip may be connected to each of the members for connecting with the vertebral rods. The grips may be rotatably positioned on the members. A connector may connect together the first and second members. The connector may provide for various manners of adjusting the position of the members about different axes. In one embodiment, the connector and the construction of the device may provide for adjustment of a length of the device.

### BRIEF DESCRIPTION OF THE DRAWINGS

FIG. 1 is a perspective view of a connection device that extends between rods according to one embodiment;

FIG. 2 is a perspective view of a first member according to one embodiment;

FIG. 3 is a perspective view of a second member according to one embodiment;

FIG. 4 is an exploded view of a connection device according to one embodiment;

FIG. 5 is a perspective view of an element of a connector according to one embodiment; and

FIG. 6 is a perspective view of a connection device according to one embodiment.

### DETAILED DESCRIPTION

The present application is directed to devices and methods to connect together first and second rods. The device includes first and second members that are adjustably connected together by a connector. The connector allows for adjustment of the members in different angular orientations. In one embodiment, the connector also allows for adjustment of the length.

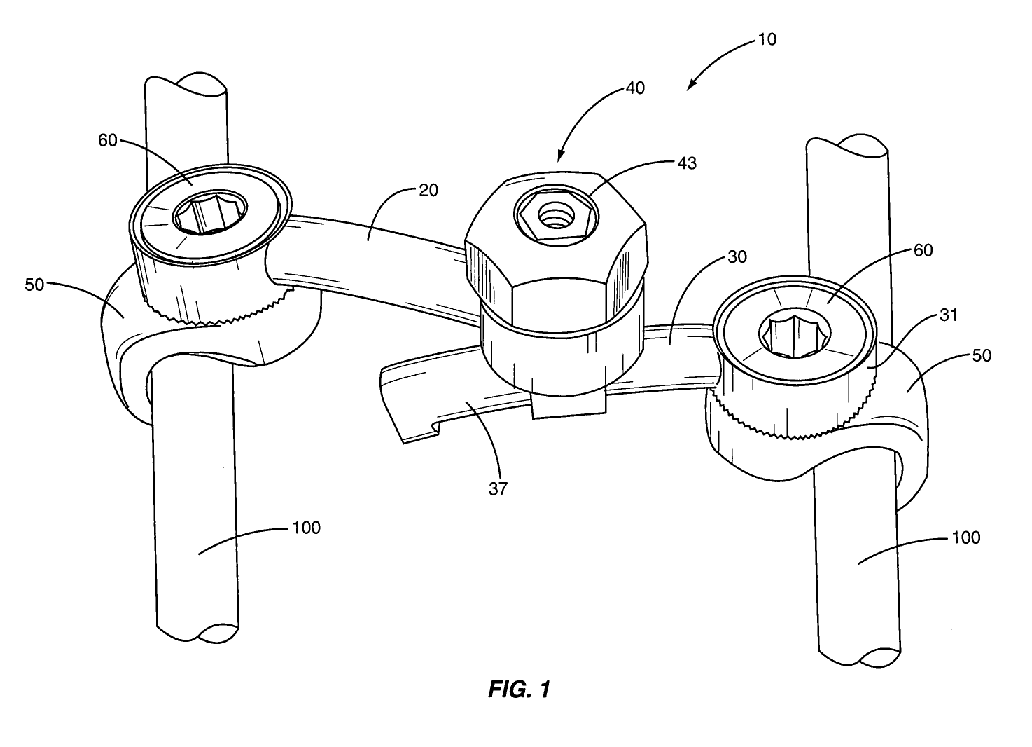

One embodiment of the device is generally illustrated in FIG. 1 and identified as element 10. This embodiment of the device 10 includes first and second members 20, 30 each having an elongated shape and being connected together by a connector 40. An outer end of each member 20, 30 includes a grip 50 to connect with a rod 100. The device 10 is adjustable to accommodate rods 100 at different positions and orientations.

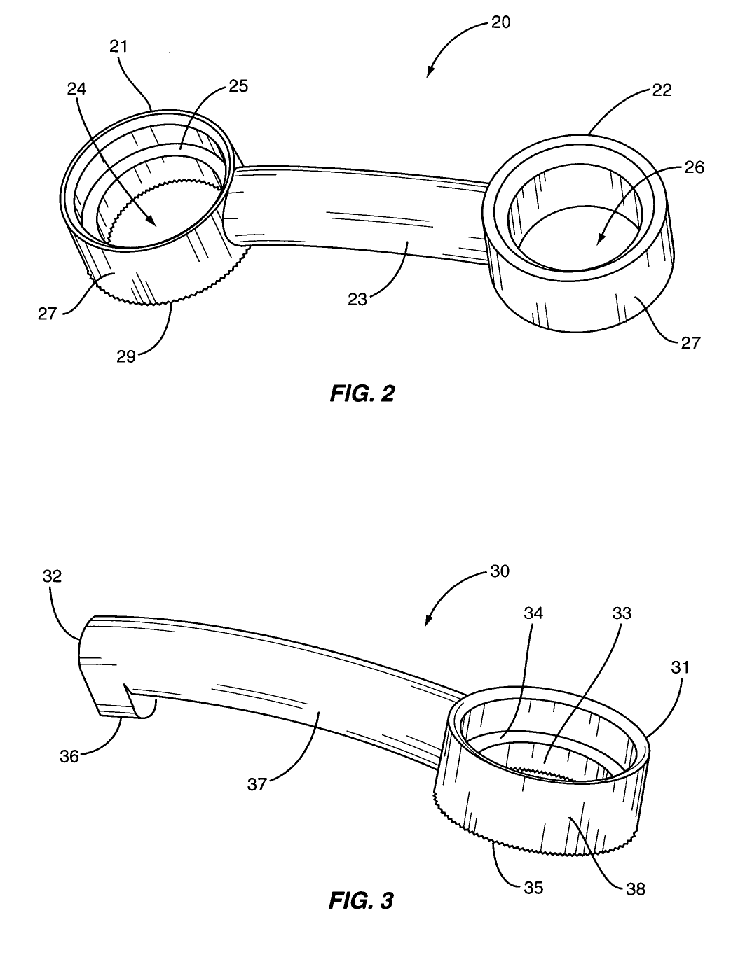

One embodiment of the first member 20 is illustrated in FIG. 2 and includes first and second ends 21, 22 separated by a rod 23. In use, the first end 21 is positioned near one of the rods 100, and the second end 22 is positioned towards a central section of the device 10. An opening 24 positioned near the first end 21 is sized to receive a fastener 60. In the embodiment as illustrated in FIG. 2, opening 24 has a substantially circular shape. Opening 24 may also have other shapes to accommodate various types and shapes of fasteners 60. Examples include polygonal shapes such as a rectangular shape, and a triangular shape. In one embodiment, opening 24 has an elongated width such that the fastener 60 is adjustably positionable at a selective location along the width.

Opening 24 illustrated in FIG. 2 is formed by a wall 27. In one embodiment, wall 27 continuously extends around the entirety of the opening 24. In another embodiment, on or more gaps extend through the wall 27. The gaps may be sized to attach and remove the fasteners 60 in a different second direction, in addition to the attachment and removal methods that are required for the fully enclosed opening 24. In one embodiment, a fully-enclosed opening 24 requires vertical fastener insertion and removal, and a gapped opening 24 can use either vertical or horizontal fastener insertion and removal. In one gapped opening embodiment, the wall 27 is constructed of a flexible, resilient material for the gap to be expanded to an enlarged size during insertion and removal of the fastener 60, and then return towards the normal, smaller size after the fastener 60 passes into or out of the opening 24.

In one embodiment, a shelf 25 extends into the opening 24 from an inner edge of the wall 27. Shelf 25 may be substantially perpendicular with the wall 27, or extend at various other angles. In the embodiment of FIG. 2, shelf 25 extends from the wall 27 around the entirety of the opening 24. In another embodiment, shelf 25 is not continuously, but rather located at limited lengths along the opening 24. Shelf 25 may act as a stop which in one embodiment contacts a head 61 of the fastener 60 as a shaft 62 extends beyond the shelf 25 and into the grip 50. In one embodiment, the opening 24 is threaded.

In one embodiment, ridges 29 are positioned on a lower face of the wall 27. Ridges 29 may be positioned across an entirety or a limited area of the lower face. Ridges 29 may be used to position the grip 50 as will be explained in detail below.

In the embodiment of FIG. 2, the first member 20 also includes an opening 26 positioned towards a second end 22 and being sized to receive the connector 40. In the embodiment of FIG. 2, opening 26 has a substantially circular shape formed by wall 27. Opening 26 may have other shapes and sizes as described above for opening 24. Outer wall 27 may further be continuous as illustrated in FIG. 2, or may have gaps as discussed above. FIG. 2 illustrates the opening 26 having a substantially smooth inner interior wall. Other embodiments may include one or more shelves 25 extending from the inner edge of the wall 27. In one embodiment, opening 26 is threaded.

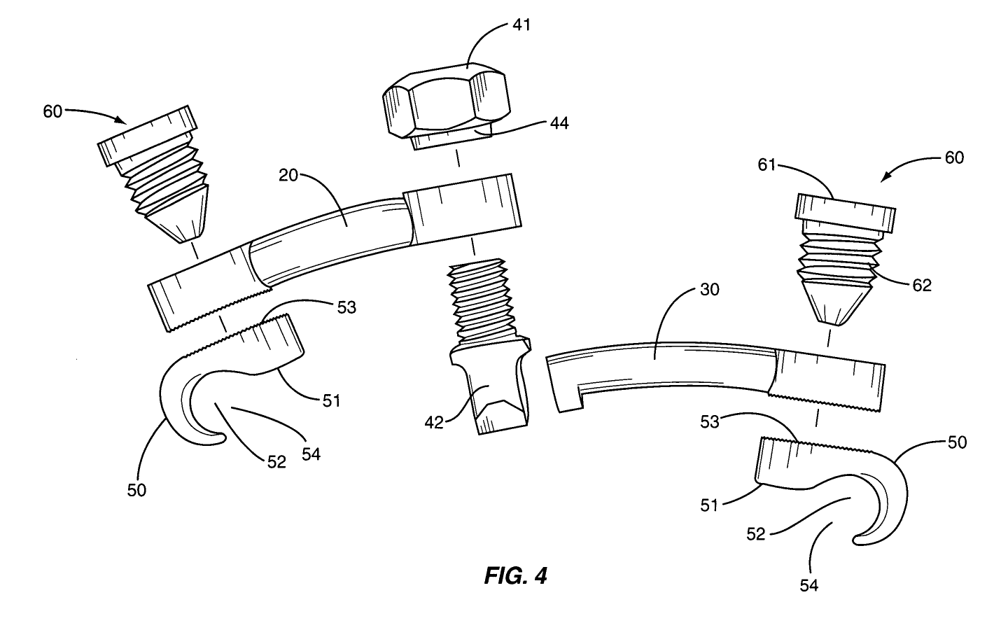

In the embodiment of FIG. 2, a rod 23 extends between the openings 24, 26. Rod 23 may have a variety of lengths and cross-sectional shapes depending upon the requirements. In one embodiment, rod 23 has a circular cross-sectional shape and includes a slight curve along the longitudinal length as illustrated in FIG. 4. Rod 23 may be constructed of a single member. In another embodiment, rod 23 is constructed of two or more members that are connected together. The members may be fixedly connected together, or may be movably connected together to allow for pivoting movement.

FIG. 2 illustrates openings 24, 26 located at the ends of the rod 23. In another embodiment, one or both of the openings 24, 26 are located along an interior section of the rod 23. In one embodiment, first member 20 includes more than two openings 24, 26. In one specific embodiment, first member 20 includes a first opening 24 positioned at an end of the rod 23, and a second opening 24 positioned inward along the rod 23. In embodiments having two or more of either openings 24, 26, openings 24, 26 may have the same shape and size, or may have different shapes and sizes.

One embodiment of the second member 30 is illustrated in FIG. 3 and includes first and second ends 31, 32 separated by a rod 37. In use, the first end 31 is positioned near a rod 100, and the second end 32 is positioned towards a central section of the device 10. An opening 33 is positioned towards the first end 31 in one embodiment and is sized to receive a fastener 60. In the embodiment of FIG. 3, opening 33 has a substantially circular shape formed by a wall 38. Opening 26 may have other shapes and sizes similar to the embodiments described above for opening 24. Further, wall may be continuous as illustrated in FIG. 3, or may have gaps as discussed above. In one embodiment, the inner edge of the opening 26 includes an outwardly-extending shelf 34 sized to contact the fastener head 61 when the fastener 60 is inserted into the opening 26. Shelf 34 may be continuous and extend around the entirety of the opening 26, or may extend around less than the entirety. In one embodiment, the inner walls of the opening 33 are threaded. In one embodiment, ridges 35 are positioned on a lower face of the wall 38 to position the grip 50.

In one embodiment as illustrated in FIG. 3, a tab 36 is positioned at the second end 32 and extends outward from the rod 37. Tab 36 may be constructed as an integral part of the rod 37, or may be constructed of a separate material that is attached to the rod 37. Tab 36 may have a variety of sizes to extend outward from the rod 37 differing amounts. Tab 36 may further be positioned at a variety of angular orientations relative to the rod 37. In one embodiment as illustrated in FIG. 3, a single tab 36 extends substantially perpendicularly outward from a lower section of the rod 37. In another embodiment, tab 36 extends outward from a side or top section of the rod 37. In one embodiment, more than one tab 36 extends outward from the rod 37.

Rod 37 may have a variety of lengths and shapes depending upon the application, including a circular cross-sectional shape as illustrated in the embodiment of FIG. 3. Rod 37 may be substantially straight, or have a slight curve along the longitudinal length. In another embodiment, rod 37 is constructed of two or more members that are connected together. The members may be fixedly connected together, or may be movably connected together to allow for pivoting movement.

The embodiment of FIG. 3 illustrates the tab 36 positioned at the second end 32 of the rod 37. In another embodiment, tab 36 is positioned along an interior portion of the rod 37 at a distance from the second end 32. In one embodiment, a first smaller tab is positioned at an interior point along the rod 37, and a second larger tab is positioned at the second end 32. The embodiment of FIG. 3 also includes the opening 33 positioned at the first end 31. In another embodiment, opening 33 is positioned along at an interior point along the rod 37 and distanced from the first end 31. In one embodiment, two or more openings 33 are positioned along the rod 37.

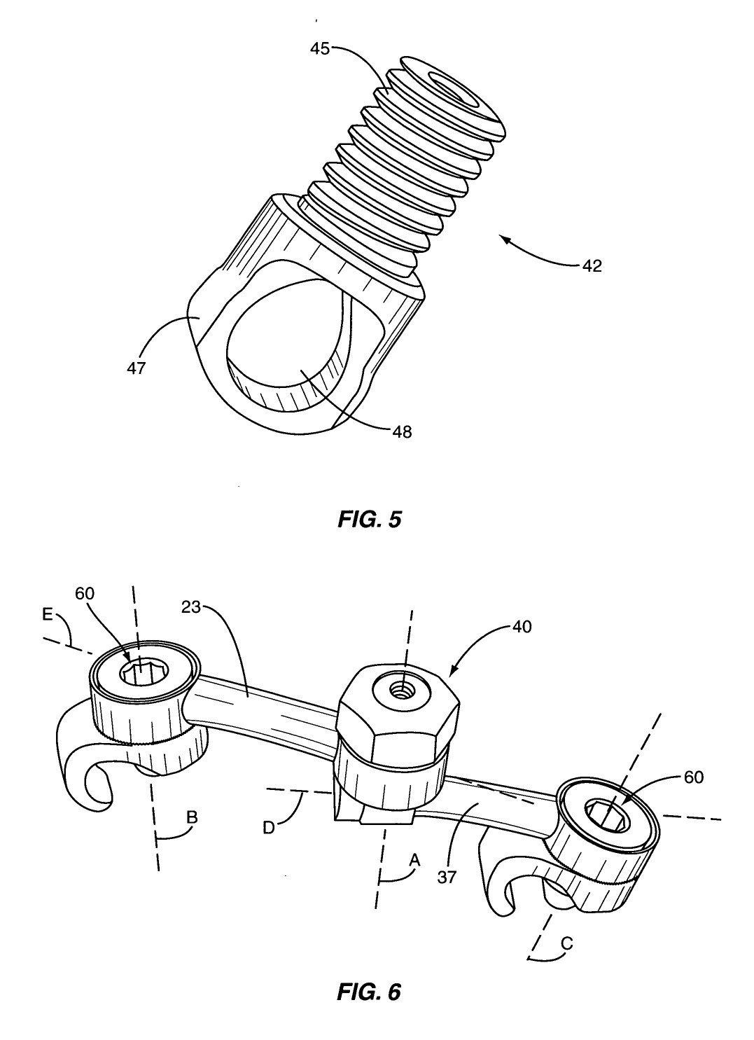

In one embodiment, connector 40 includes a first member 41 and a second member 42 as illustrated in FIGS. 1, 4, and 5. First member 41 in this embodiment is a locking nut having a threaded opening 43. In one embodiment, a neck 44 extends outward from a lower surface of the first member 41 and seats within the opening 26 in the member 20. In one embodiment, second member 42 includes a threaded shaft 45 that extends from a head 47. The shaft 45 may be sized to fit within the opening 26 of the member 20 and mate with the threaded opening 43 of the first member 41. In the embodiment of FIG. 5, the entire shaft 45 is threaded. In other embodiments, a limited portion of the shaft 45 is threaded, with one specific embodiment featuring an end of the shaft 45 being threaded that is opposite from and distanced from the head 47. In yet another embodiment, the shaft 45 is not threaded.

In one embodiment, second connector member 42 is constructed to receive the rod 37. In one embodiment as illustrated in FIG. 5, second member 42 includes an opening 48 sized to receive the rod 37. In one embodiment, opening 48 is shaped and size to substantially match the shape and size of the rod 37. In one specific embodiment, the shape of both the opening 48 and the rod 37 is substantially circular. In one embodiment, opening 48 has an elongated shape. In one embodiment, the opening 48 is formed within the head 47 and encloses the opening 48. In another embodiment, a gap extends through the head 47 and into the opening 48. The gap is sized such that the rod 37 can be laterally inserted and removed from the opening 48. In one embodiment, head 47 has a curved orientation similar to the grip 50 and having a receiving section for holding the rod 37. In one gapped embodiment, the head 47 is constructed of a flexible, resilient material that expands as the rod 37 passes through the gap and rebounds towards a smaller size after rod passage. In one embodiment, opening 48 has a larger cross-sectional size than the rod 37. This provides for the rod 37 to move within the opening 48 prior to tightening of the connector 40 as will be explained in detail below.

Grips 50 include in one embodiment a fastening section 51 and a receiving section 52. Fastening section 51 includes an opening in one embodiment sized to receive a shaft 62 of the fastener 60 as explained in detail below. Receiving section 52 is sized to receive the rod 100. In one embodiment, receiving section 52 includes an inlet 54 through which the rod 100 is inserted. In one embodiment, the size of the inlet 54 is greater than the diameter of the rod 100 such that the rod 100 can be laterally inserted into and removed from the receiving section 52. In another embodiment, receiving section 52 is enclosed by the grip 50 and requires the rod 100 be inserted in an axial direction.

In one embodiment, ridges 53 are positioned along top surface of the fastening section 51. Ridges 53 may extend over a limited area of the top surface, or may cover the entirety of the top surface. Ridges 53 are sized to complement ridges 29 on the lower surfaces of the first and second members 20, 30.

FIG. 1 illustrates one embodiment of an assembled device 10 with the connector 40 connecting the first member 20 to the second member 30. In the assembled orientation of this embodiment, the rod 37 of the second member 30 is positioned within the opening 48 of the connector 40 with the tab 36 positioned on an opposite side of the connector 40 from the first end 31. The assembled device 10 of this embodiment is further oriented with the fasteners 60 connecting the grips 50 to first and second members 20, 30. Fasteners 60 extend through the opening 24, 33 of the members 20, 30 and into the grips 50. In one embodiment, the ends of the fastener shafts 62 extend into the receiving section 52 and across at least a portion of the inlet 54 to maintain the rods 100 within the receiving sections 52 and connected to the grips 50. In one embodiment, further insertion of the fasteners 60 into the openings 24, 33 causes the shafts 62 to extend further into the receiving section 52 and clamp the rod to the grip 50.

FIG. 6 illustrates the adjustable aspects that can be accommodated by one embodiment. A first adjustment of this embodiment features relative movement of the first and second members 20, 30 about axis A. One method of adjusting the device about axis A includes orienting the connector members 41, 42 to allow for the second member 42 and attached second rod member 30 to rotate relative to the first rod member 20. In one embodiment, the second rod member 30 may rotate up to about 330° relative to the first rod member 20. In one embodiment, connector 40 includes a spacer (not illustrated) positioned between members 41, 42 to space the first rod member 20 away from the second rod member 30. This spacing prevents contact between the rod members 20, 30 and allows for about 360° of rotation about axis A.

In one embodiment, rotation of the members 20, 30 about axis A is accomplished by unlocking the first connector member 41 from the second connector member 42. In this unlocked orientation, the members 20, 30 may be rotated to the desired positions. Once at a proper angular position in this embodiment, connector 40 is moved to a locked orientation to prevent further relative movement between the members 20, 30. In one embodiment, moving the connector 40 to the unlocked orientation comprises rotating the first member 41 in a first direction along the threads on the shaft 45 of the second member 42 and loosening the connection. In this embodiment, moving the connector 40 to the locked orientation comprises rotating the first member 41 in a second direction along the threads on the shaft 45 of the second member 42 and tightening the connection. In one embodiment, moving the connector 40 towards the locked orientation causes the second member 42 to move into the first member 41. This movement may cause the rod 37 within the opening 48 to move into contact with a lower surface of the wall 27 of the first member 20. Continuing rotation of the first member 41 along the threads of the second member 42 increases the amount of force applied to the rod 37.

The connection device 10 of the embodiment of FIG. 6 further accommodates adjustments about axes B and C. Fasteners 60 attach a grip 50 to each of the members 20, 30. In one embodiment, grips 50 are rotatably connected to the members 20, 30 to rotate up to 360° to receive and maintain the rods 100. Once the grips 50 are positioned at the proper rotational position in one embodiment, the fasteners 60 are moved from an unlocked to locked orientation to fixedly connect the grips 50 to the rod members 20, 30 and prevent further movement. FIG. 6 illustrates a configuration of one embodiment with a first grip 50 (i.e., the left grip as it appears in the Figure) extending outward substantially away from the connector 40. A second grip 50 (i.e., the right grip) is rotated and positioned substantially under rod 37. In some embodiments, grips 50 can face in a combination of directions, and may even face in the same direction.

In one embodiment, the second member 30 may be rotated about axis D. Rod 37 in one embodiment is sized to rotate relative to the connect 40. In one specific embodiment, opening 48 of the connector 48 is larger than the rod 37 thus allowing for the rod 37 to be rotated within the opening 48. In one embodiment, rod 37 rotates through angular orientations about axis D of about 240°. In one embodiment, tab 36 on the rod 37 contacts the connector 40 and/or the first member 20 to prevent further rotation. In one embodiment, the size of the tab 36 is reduced to allow for greater angular orientations. In one embodiment, tab 36 is removed from the rod 37 allowing for rotation about axis D of about 360°. In one embodiment, connector 40 can be moved to a locked orientation to prevent further rotation.

In one embodiment, a length of the device 10 can be adjusted to accommodate rods 100 at different spacings. The length is defined as the distance between receiving sections 52 of the grips 50. In one embodiment, a method of adjusting the length includes adjusting the rotational position of the members 20, 30 about axis A. In one embodiment, the length may be adjusted by adjusting the rotational positions of the grips 50 about axes B and C.

In one embodiment, length adjustment is accomplished by moving the second member 30 relative to the connector 40. In one embodiment, rod 37 is sized to move within the opening 48 in the connector 40. A maximum length in this embodiment occurs when a second end 32 is positioned at the connector 40. In one specific embodiment, tab 36 extending from the second end 32 is in contact with the head 47 of the connector 40. In one embodiment, tab 36 has a larger size than the connector opening 48 thus preventing the rod 37 from pulling through the opening 48 and detaching from the connector 40. Shorter lengths are accomplished in this embodiment by moving the rod 37 inward through the opening 48 such that the first end 31 is positioned in closer proximity to the connector 40. Once positioned at the correct length, connector 40 is moved to a locked orientation to prevent further movement of the rod 37 within the opening 48. In one embodiment, tab 36 is sized to fit through the opening 48 when the rod 37 is oriented in a predetermined rotational position relative to the connector 40. In one embodiment, tab 36 fits within opening 48 when the rod is turned upside down (i.e., rotated by about 180 degrees) relative to the connector 40. Once the tab 36 is inserted within the opening 48, rod 37 is returned towards the normal orientation which prevents removal of the tab 36 from the opening 48. In one embodiment, tab 36 and opening 48 each have a substantially teardrop shape.

In one embodiment, each of the adjustments described above may be performed independently. By way of example, device 10 may be adjusted about axis A regardless of the length or the rotational position about axes B, C, or D.

FIGS. 1 and 4 illustrate one embodiment having ridges 29, 35, 53 positioned on the engaging surfaces of the grips 50 and members 20, 30. The ridges 53 of the grips 50 complement the ridges 29, 35 on the members 20, 30 to engage together when the fastener 60 is tightened. In one embodiment, ridges are positioned on the connector 40 and second end 22 of the first member 20 to engage when the connector 40 is in a locked orientation. In one embodiment, ridges comprise sets of peaks and valleys positioned on an entirety or a portion of the engagement surfaces. Other ridge designs are also contemplated and may include knurled surfaces, coarse, abrasive or frictional surface features and gear-type surfaces.

Rods 23, 37 of the first and second members 20, 30 may be substantially straight or may be curved. In one embodiment, straight rods 23, 37 result in the axes A, B and C being substantially parallel. When one or both rods 23, 37 are curved, axes A, B, C are non-parallel. Rods 23, 37 may have the same shape and length, or may have different shapes and/or lengths. In one embodiment, axis D is substantially perpendicular to one or more of axes A, B, and C.

In another embodiment, first rod member 20 has a second end similar to the second rod member 30. In this embodiment, first rod member 20 includes a tab 36 and is adjustably mounted to the connector 40.

In one embodiment, connector 40 connects the first and second members 20, 30 in a vertical orientation (e.g., member 20 is above member 30). In this embodiment, members 20, 30 overlap a limited amount or have no vertical overlap in an extended elongated orientation, and have a greater amount of vertical overlap in a reduced length orientation. In another embodiment, connector 40 connects the members 20, 30 in a horizontal orientation (e.g., member 20 is on a lateral side of member 30). This orientation may result in no vertical overlap, but rather results in horizontal overlap.

In one embodiment as illustrated in FIG. 6, the first end 21 of the first member 20 is adjustable about axis E. In one embodiment, the first end 21 is movably connected to the rod 23 such that the rod 23 remains relatively stationary as the first end 21 rotates. In another embodiment, rod 23 is movably connected to the second end 22. In this embodiment, the first end 21 and rod 23 rotate together as the second end 22 remains relatively stationary. Movement of the first end 21 provides another adjustable feature for accommodating vertebral rods 100 at various orientations.

Spatially relative terms such as “under”, “below”, “lower”, “over”, “upper”, and the like, are used for ease of description to explain the positioning of one element relative to a second element. These terms are intended to encompass different orientations of the device in addition to different orientations than those depicted in the figures. Further, terms such as “first”, “second”, and the like, are also used to describe various elements, regions, sections, etc and are also not intended to be limiting.

The present invention may be carried out in other specific ways than those herein set forth without departing from the scope and essential characteristics of the invention. FIG. 1 illustrates one embodiment with the grips 50 positioned on a lower surface of the rod members 20, 30. One or both grips 50 may also be positioned on an upper surface of the rod members 20, 30. The present embodiments are, therefore, to be considered in all respects as illustrative and not restrictive, and all changes coming within the meaning and equivalency range of the appended claims are intended to be embraced therein.

What is claimed is:

1. A device to connect together first and second vertebral rods comprising:
a first elongated member having a first curved portion to connect to the first vertebral rod, the first curved portion being rotatably coupled to the first member about a first axis;
a second elongated member having a second curved portion to connect to the second vertebral rod, the second curved portion being rotatably coupled to the second member about a second axis; and
a connector connecting the first and second members in an adjustable manner to adjust a combined length of the first and second members;
the connector providing rotational movement between the first and second members about a third axis and a fourth axis, the third axis being substantially perpendicular to the fourth axis.

2. The device of claim 1, wherein the first and second axes are substantially parallel.

3. The device of claim 1, wherein the first and second curved portions can rotate about 360 degrees relative to the first and second members.

4. The device of claim 1, wherein the combined length of the first and second members is defined along the third axis when the first and second elongated members are aligned in a vertically overlapping configuration.

5. The device of claim 4, wherein the first and second axes are substantially perpendicular to the third axis.

6. The device of claim 1, wherein the first, second, and fourth axes are substantially parallel.

7. The device of claim 1, wherein the connector is fixedly attached to one of the first and second members.

8. The device of claim 1, wherein the connector positions the first and second elongated members in a vertically overlapping configuration.

9. The device of claim 1, wherein the first curved portion is rotatably connected to the first member and rotatable up to about 360 degrees about the first member.

10. A device to connect together first and second vertebral rods comprising:
first and second elongated members;
a first curved portion rotatably mounted to the first member and sized to connect to the first vertebral rod;
a second curved portion rotatably mounted to the second member and sized to connect to the second vertebral rod; and
a connector connecting together the first and second members, the connector rotatably connecting together the first and second members about first and second axes, the connector further connecting together the first and second members to adjust a length between the attachment devices.

11. The device of claim 10, wherein the second axis is substantially perpendicular to the first axis.

12. The device of claim 10, wherein the connector is fixedly attached to the first member and movably attached to the second member.

13. The device of claim 10, wherein the first and second curved portions are attached to outer ends of the first and second members.

14. The device of claim 13, wherein the connector is fixedly attached to an inner end of one of the first and second members.

15. The device of claim 10, wherein the first curved portion is rotatable about a third axis and the second curved portion is rotatable about a fourth axis, the third and fourth axes being substantially parallel with the first axis.

16. The device of claim 10, wherein the first member comprises a rod extending between first and second openings, the first opening sized to receive the connector and the second opening sized to attach with the first curved portion.

17. The device of claim 10, wherein the second member comprises a rod and an opening at one end of the rod, the opening is sized to attach with the second curved portion.

18. The device of claim 17, wherein the rod extends through an opening in the connector and is sized to axial move within the opening, the rod further comprising a tab to prevent the rod from detaching from the connector.

19. A device to connect together first and second vertebral rods comprising:
a first elongated member having a first curved portion that connects to the first vertebral rod, the first curved portion being rotatable about a first axis;
a second elongated member having a second curved portion that connects to the second vertebral rod, the second curved portion being rotatable about a second axis;
a connector connecting the first and second members in an adjustable manner to adjust a combined length of the first and second members;
the connector providing rotational movement between the first and second members about a third axis and a fourth axis.

20. The device of claim 19, wherein the first and second axes are substantially parallel.

21. The device of claim 19, wherein the first, second, and fourth axis are substantially parallel.

22. The device of claim 19, wherein the third axis is substantially perpendicular to the fourth axis.

23. The device of claim 19, wherein the connector further comprises a spacer, the first elongated member being rotatable relative to the second elongated member by about 360 degrees about the fourth axis.

24. A device to connect together first and second vertebral rods comprising:
a first extension member;
a second extension member; and
a connector connected to each of the first and second extension members, the connector fixedly connected to the first extension member and comprising a receiving section to adjustably receive the second extension member;
a first curved portion to connect with the first vertebral rod and being rotatably connected to the first member about a first axis; and
a second curved portion to connect with the second vertebral rod and being rotatably connected to the second member about a second axis;
the second extension member being positioned to rotate about third and fourth axes.

25. The device of claim 24, wherein the fourth axis extends through a center of the connector.

26. The device of claim 24, wherein the second extension member includes a rod section and a tab, the rod section being sized to pass through the receiving section and the tab being sized to prevent passage through the receiving section.

27. The device of claim 26, wherein the tab is positioned at an inner end of the second extension member.

28. The device of claim 24, wherein the second extension member is axially movably within the connector to adjust a combined length of the first and second extension members.

29. A device to connect together first and second vertebral rods comprising:
first and second extension members having inner and outer ends separated by an interior section, each of the outer ends having rotatable curved portions with receiving sections to respectively connect with one of the first and second vertebral rods, the second extension member having a tabbed section positioned towards the inner end; and
a connector connecting together the first and second extension members to be movable about a first axis, the connector having an opening sized to receive the second extension member, the opening sized to receive the interior section of the second extension member and permit axial movement of the second extension member through the opening to adjust a combined length and rotational movement to adjust an angular position about a second axis, the opening being smaller than the tabbed section to prevent detachment of the second extension member from the connector.

30. The device of claim 29, wherein the first axis is substantially perpendicular to the second axis.

31. The device of claim 29, the second extension member has a circular cross sectional shape.

32. The device of claim 29, wherein the connector comprises a locking member and a receiving member, the receiving member having the opening.

33. A method of connecting together first and second vertebral rods comprising the steps of:
rotating a first curved portion about a first axis and connecting the first curved portion to the first vertebral rod;
rotating a second curved portion about a second axis and connecting the second curved portion to the second vertebral rod;
adjusting a connector that connects the first and second elongated members to an adjustable state and adjusting a combined length of the first and second elongated members;
while the connector is in the adjustable state, adjusting a relative position of the first and second elongated members about a third axis;
while the connector is in the adjustable state, adjusting a relative position of the first and second elongated members about a fourth axis; and
adjusting the connector to an unadjustable state and fixing the first and second elongated members.

34. The method of claim 33, wherein the step of adjusting the combined length of the first and second elongated members comprises sliding one of the first and second elongated members relative to the connector.

35. The device of claim 33, further comprising preventing the combined length from exceeding a predetermined amount by contacting a tab on one of the first and second elongated members with the connector.

36. A method of connecting together first and second vertebral rods comprising the steps of:
adjusting a first curved portion angular position about a first axis and connecting the first curved portion to the first vertebral rod;
adjusting a second curved portion angular position about a second axis and connecting the second curved portion to the second vertebral rod;
adjusting a third angular position about a third axis, the third angular position being a third angle formed between a first member connected to the first curved portion relative to a second member connected to the second curved portion;
adjusting a fourth angular position about a fourth axis, the fourth angular position being a fourth angle formed between the first member and the second member; and
adjusting a combined length of the first and second members.
